SEQUENCE LISTING (9 sequences)

SEQ ID NO: 1 - DNA, 2001 bp, Homo sapiens
agttggaatgtatttatggcaggttagtgtatcctggatacagtatgtccagaaaaggactcttaagataaatggtaaattgaagttaagttgctgctctatgctacattttcttttcagccaggtaacaagtattttgggctatctactatggtgctgatgttgactgagtgacctgctagggcccaagggagaagcagcaagagtaagacccttggccaggcacagtggctcctgcctgtaaacccagcactttgggaggccaagaagggaggatcacttgaggccaggagttcagcctaggcaacatagcaagacctcatctctacaaaataaaaaattagacaggtgtagtggtgcaagtctgcagtcccagctacttgcaaggctgaggtgggaggattgcttgagcacaggattttgaatcagccagggcaacctagcaagaccccatctccacaaaaaaaaattttacaagaccctgggagcagagagcatggcctgggccctcttacctggcactgtggggtcttactctgtgtcaccctcagtgggcatcaggggagagattgtgccaagacactgagctgggccaggggcagattctgccttgcaggcaggacctcagcccgaagccacatcttcctccaggacagtttctatctgcagaaattaggaaatggtctaagtctttatcccagacatcccagaactggaatccagcctcttttatcaggttttatgggctgttatcagaggccagagggatcactcaccaacagagcccagctggcatttcccactgggcacccacggcgaccccaacaaggcagaggagagtgtagactctggcttgtccacaccagcccagtcacttctgctgttggttacagcatggcttataccagcaaagagctcggttttcagaagcaggcagaaccagcaagtcccaaggttggaggtagaactctaaaatggaagagctggggaaacagggtgctcctcctctccttgcctctctccatcttcccctagagttgggtgctgtggagttctctgctcttcacaacaaaaaaacagctgggcatggtggtgtgcacctgtggtccaggaggagccgtgattgcaccactgcactccagcctgggccaccgagcaagaccccatctctaaaaacataaaagtaaaaataaaccaacctaggtcctgagacccagtgctggaggaccagaagctatagccttatgtttccgcagttgttgttttttttcttttttgagatggagtctcactctcactctgtcacccaggctggagtgcagtggcacaatcttggctcactgcaacctccacctcctgggtgcaagtgattctcctgcctcagcctcctgagtagctgggattacaggcgtgagccaccatgcctggctgatttttgtatttttagtagagatggagtttcaccacgttggccaggctggtctcgaactcctgaccttagatgatccactcgcccagccacatttcctcctaagggtcttcctaactgtggtgactacgtattgtgggcagccctcatttaaaacaatctgtctatgctttttgttctggaaaatatggacaacataactacacaagttatgatctagccaaagtgcttctgaattaccaaattatggggtttcattagaagaaatgacagcgataacatgattaataggagtttttttagcagaatgccctcatgttaagccttccgaagcctttcttttaaacccctagtttttctcgcaatgatgttatttttgttttttaatatggaagactagaggttactctcagtcaaaaaaatatgaaacagttcactaagacccacagaaaaccctaccgtctgcttctcctacagagcgtttaattagtatttcctagataaggaacgtagatggtcgtggtaaaagacagctattttcaggcacggtttctcgtgtgctttaattacag

SEQ ID NO: 2 - DNA, 7 bp, Homo sapiens
ccgaugg

SEQ ID NO: 3 - DNA, 23 bp, Artificial
  Synthetic construct
gctattttcaggcacggtttctc

SEQ ID NO: 4 - DNA, 21 bp, Artificial
  Synthetic construct
tccacattgttggggtcgttc

SEQ ID NO: 5 - DNA, 21 bp, Artificial
  Synthetic construct
agaaaggccgagaacccttca

SEQ ID NO: 6 - DNA, 22 bp, Artificial
  Synthetic construct
cgacggtccccttctggaatgg

SEQ ID NO: 7 - DNA, 15 bp, Artificial
  Synthetic construct
caagcgctgagtccc

SEQ ID NO: 8 - DNA, 38 bp, Artificial
  Synthetic construct
actgctcgagcggccgcttttagcagaatgccctcatg

SEQ ID NO: 9 - DNA, 21 bp, Artificial
  Synthetic construct
gtttttggtgacgggtatggc

(19) United States
(12) Patent Application Publication
(10) Pub. No.: US 2006/0275843 A1
(43) Pub. Date: Dec. 7, 2006

(54) Methods for diagnosing the presence or stage of cancer

(76) Inventors: Alain Nepveu, Montreal, CA (US); Brigitte Goulet, Montreal (CA); Nam-Sung Moon, Medford, MA (US); Matthew Bogyo, Mill Valley, CA (US); Amos Baruch, San Francisco, CA (US); Peter Watson, Winnipeg (CA)

Correspondence Address: STERNE, KESSLER, GOLDSTEIN & FOX PLLC, 1100 NEW YORK AVENUE, N.W., WASHINGTON, DC, 20005, US

(21) Appl. No.: 10/535,156

(22) PCT Filed: Nov. 13, 2003
(86) PCT No.: PCT/US03/36341

§ 371 Date: Apr. 5, 2006

(30) Foreign Application Priority Data
Nov. 14, 2002 (US) 60426293

(51) Int. Cl. G01N 33/574

(52) U.S. Cl. 435/7.23

(57) ABSTRACT

The present invention provides antibodies to truncated isoforms of CCAAT-displacement protein/Cut homeobox (CDP/Cux) which are useful in diagnostic and prognostic methods for detecting cancer. Further provided are methods for detecting RNA transcripts encoding p75 for the detection of cancer.

### BACKGROUND OF THE INVENTION

CCAAT-displacement protein/cut homeobox (CDP/Cux) belongs to a family of transcription factors present in all metazoans and is involved in the control of proliferation and differentiation (Nepveu (2001) Gene 270:1-15). In Drosophila melanogaster, a large number of phenotypes result from the insertion of transposable insulator sequences that interfere with the function of tissue-specific enhancers of Cut, a CDP/Cux homolog (Jack, et al. (1991) Development 113:735-747; Jack and DeLotto (1995) Genetics 139:1689-1700; Modolell, et al. (1983) Proc. Natl. Acad. Sci. USA 80: 1678-1682; Cai and Levine (1997) EMBO J. 16:1732-1741; Dorsett (1993) Genetics 134:1135-1144). The affected tissues include the wings (“cut wing”), legs, external sense organs, Malpighian tubules, tracheal system and some structures in the central nervous system (Jack, et al. (1991) supra; Jack (1985) Cell 42:869-876; Jack and DeLotto (1992) supra; Liu, et al. (1991) Genetics 127:151-159; Liu and Jack (1992) Dev. Biol. 150:133-143; Bodmer, et al. (1987) Cell 51:293-307; Blanc (1942) Univ. Calif. Publ. Zool. 49; Braun (1942) J. Exp. Zool. 84: 325-350; Hertweck (1931) J. Exp. Zool. 139:559-663). Humans have two CDP/Cux genes, CDP-1 and CDP-2, as do mouse and chicken, Cux-1 and Cux-2 (Neufeld, et al. (1992) supra; Valarche, et al. (1993) Development 119:881-896; Quaggin, et al. (1996) J. Biol. Chem. 271:22624-22634). While Cux-2 is expressed primarily in nervous tissues, Cux-1 is present in most tissues (Neufeld, et al. (1992) supra; Andres, et al. (1992) Development 116:321-334; Ellis, et al. (2001) Genes Dev. 15:2307-2319). Cux-1 knockout mice display phenotypes in various organs including curly whiskers, growth retardation, delayed differentiation of lung epithelia, altered hair follicle morphogenesis, male infertility, and a deficit in T and B cells (Ellis, et al. (2001) supra; Sinclair, et al. (2001) Blood 98:3658-3667; Luong, et al. (2002) Mol. Cell. Biol. 22:1424-1437; Tufarelli, et al. (1998) Dev. Biol. 200:69-81). In contrast to the small size of the cux-1 knockout mice, transgenic mice expressing Cux-1, under the control of the CMV enhancer/promoter, display multi-organ hyperplasia and organomegaly (Ledford, et al. (2002) Dev. Biol. 245: 157-171). Thus, genetic studies both in Drosophila and mice indicate that the CDP/Cux/Cut gene plays an important role in the development and homeostasis of several tissues.

In tissue culture, the expression and activity of CDP/Cux has been associated with cellular proliferation (Holthuis, et al. (1990) Science 247:1454-1457; van Wijnen, et al. Proc. Natl. Acad. Sci. USA 88:2573-2577; Coqueret, et al. (1998) EMBO J. 17:4680-4694), the repression of genes turned on in terminally differentiated cells (Pattison, et al. (1997) J. Virol. 71:2013-2022; Lawson, et al. (1998) Blood 91:2517-2524; van Gurp, et al. (1999) Cancer Res. 59:5980-5988; O'Connor, et al. (2000) J. Virol. 74:401-410; Skalnik, et al. (1991) J. Biol. Chem. 266:16736-16744; Teerawatanasuk, et al. (1999) J. Neurochem. 72:29-39), and regulation of matrix attachment regions (Liu, et al. (1997) Mol. Cell. Biol. 17:5275-5287; Banan, et al. (1997) J. Biol. Chem. 272:18440-18452; Chattopadhyay, et al. (1998) J. Biol. Chem. 273: 29838-29846; Wang, et al. (1999) Mol. Cell Biol. 19:284-295; Stunkel, et al. (2000) J. Virol. 74:2489-2501). CDP/Cux/Cut proteins contain DNA binding domains. All proteins contain at least a Cut homeodomain (HD) and as many as three Cut repeats (CR1, CR2 and CR3). The cut superclass of homeobox genes has been divided into three classes: CUX, ONECUT and SATB (Burglin and Cassata (2002) Int. J. Dev. Biol. 46:115-123). While the Drosophila Cut, human CDP and mouse Cux genes contain three Cut repeats, in each species there is also a ONECUT gene containing a single Cut repeat (Neufeld, et al. (1992) supra; Valarche, et al. (1993) Development 119:881-896; Blochlinger, et al. (1988) Nature 333:629-635; Lemaigre, et al. (1996) Proc. Natl. Acad. Sci. USA 93:9460-9464; Lannoy, et al. (1998) J. Biol. Chem. 273:13552-13562). SATB1 includes two Cut repeat-like domains and a divergent Cut-like homeodomain (Dickinson, et al. (1997) J. Biol. Chem. 272:11463-11470).

Individual Cut repeats cannot bind to DNA on their own but need to cooperate with a second Cut repeat or with the Cut homeodomain (Moon, et al. (2000) J. Biol. Chem. 275:31325-31334). Two CDP/Cux DNA binding activities have been reported in cells. CDP/Cux p200 binds transiently to DNA, similar to the CR1CR2 domains, and carries the CCAAT-displacement activity (Neufeld, et al. (1992) supra; Skalnik, et al. (1991) supra; Moon, et al. (2000) supra;

Barberis, et al. (1987) Cell 50:347-359). At the G1/S transition of the cell cycle, proteolytic cleavage of p200 generates CDP/Cux p110, which contains CR2CR3HD and exhibits distinct DNA binding specificity and kinetics (Moon, et al. (2001) Mol. Cell. Biol. 21:6332-6345). In particular, p110 is able to make a stable interaction with DNA. Furthermore, the p110 isoform is expressed at higher levels in uterine leiomyomas (Moon, et al. (2001) supra).

Current prognostic markers for breast cancer are insufficient only 70% of patients with good prognosis breast cancer are actually cured by surgery alone while 30% have recurrence of cancer. Moreover, the establishment of lymph node status, which is one of the most useful prognostic factors in breast cancer, is not without complications. For example, lymphedema, a potentially devastating complication of axillary node dissection, may occur in up to 24% of patients. Hence, there is a need for improved diagnostic, prognostic and predicative indicators for breast tumors.

A novel CDP/Cux isoform, p75, has now been found that is encoded by mRNA initiated within intron 20 of the CDP/Cux locus. This novel isoform displays DNA binding properties distinct from that of p200, p110 and p100 CDP/Cux isoforms. While expression of the mRNA initiated in intron 20 is restricted to certain tissues or cells, the expression is activated in breast tumor cell lines and in primary human breast tumors as well as other cancerous tissue.

### SUMMARY OF THE INVENTION

The present invention provides methods for diagnosing cancer by detecting the levels of CDP/Cux isoforms in a sample isolated from a subject having or suspected of having cancer. Increased levels of truncated CDP/Cux isoforms is indicative of the presence or stage of cancer.

One embodiment provides using an antibody binding assay to detect the level of a truncated CDP/Cux isoform in a sample and comparing said levels to a known standard.

Another embodiment provides evaluating the level of p75 transcript in a sample and comparing said level to a known standard.

A kit for detecting the truncated CDP/Cux isoforms in a sample is also provided.

These and other aspects of the present invention are set forth in more detail in the following description of the invention.

### DETAILED DESCRIPTION OF THE INVENTION

The present invention provides a method of diagnosing the presence or stage of cancer by detecting the level of a truncated CDP/Cux isoform. Truncated isoforms of CDP/Cux are typically proteolytically processed isoforms of p200, i.e., p200 is cleaved by a protease to produce less than full-length isoforms. In particular, the p100 and p110 truncated isoforms are contemplated as is the newly discovered p75 isoform which is encoded by mRNA initiated within intron 20 of the CDP/Cux locus.

The p75 isoform was identified in RNase mapping analysis using a riboprobe containing exons 19, 20 and 21 of the CDP-Cux locus. A smaller protected fragment than anticipated was generated with RNA samples from certain sources, notably HeLa cells and placenta. This result indicated the existence of an alternative CDP/Cux transcript that contained exon 21, but not exon 20. This RNA transcript encoding p75 is also referred to herein as I20-mRNA. The 5′ end of the novel transcript was cloned from placenta by the method of rapid amplification of cDNA ends (RACE) using as reverse primers two successive oligonucleotides from exon 21. DNA sequencing analysis indicated that the RACE-amplified sequence upstream of exon 21 originated from intron 20 and extended at least 500 nt upstream of the intron 20/exon 21 junction. To exclude the possibility that contaminating genomic DNA may have served as a template in the RACE reaction, reverse-transcriptase polymerase chain reaction (RT-PCR) analysis was performed using as forward primers oligonucleotides from exon 19 or intron 20 and as a reverse primer, an oligonucleotide from exon 22. First-strand cDNA preparations derived from different adult human tissues (Clonetech, Palo Alto, Calif.) were used as a source of material. Fragments of 609 bp, using exon 19 and 22 primers, and 474, using intron 20 and exon 22 primers, were obtained. The latter corresponds to the size predicted for a mRNA containing sequences from intron 20, exon 21 and exon 22. This result confirmed the existence of a CDP/Cux mRNA that initiates upstream of exon 21 and does not contain exon 20. RT-PCR analysis of RNAs isolated from mouse tissue confirmed that a similar transcript was expressed at higher levels in the placenta and thymus of the mouse. The mouse I20-mRNA was expressed in mature and immature T cells, but at a higher level in mature CD4+ than in mature CD8+ T cells. Therefore, these findings indicate that the I20-mRNA is expressed in a tissue and cell-type specific manner.

RT-PCR products of the expected sizes were obtained with forward primers situated approximately 500, 1500, 2500 nt upstream of exon 21. No product was obtained with oligonucleotides positioned 3000 and 3500 nt upstream of exon 21. RNase mapping analysis was then performed with a riboprobe containing nucleotides −2270 to −2978 upstream of exon 21. A unique, protected fragment of approximately 200 nucleotides was observed, indicating that transcription of I20-mRNA starts at a position approximately 2.5 kbp upstream of the intron 20/exon 21 junction. Nucleic acid sequences corresponding to this region of intron 20 are provided as SEQ ID NO:1. These results define a novel CDP/Cux mRNA that is expressed in a tissue-specific manner and is initiated upstream of exon 21.

The I20-mRNA contains a long 5′-untranslated sequence followed by an open reading frame starting at the beginning of exon 21. An AUG codon is present at a position corresponding to nt 3224 of the HSCDP cDNA sequence (Accession No. M74099). The sequence at this position, CCGAUGG (SEQ ID NO:2), does not conform to the Kozak consensus. Yet, a protein was expressed in an in vitro transcription/translation system, and replacement of AUG for ULC completely eliminated translation. Transfection of NIH3T3 cells with an expression vector harboring nucleic acid sequences encoding mouse I20-mRNA gave rise to a novel protein of 75 kDa that co-migrated with a protein present in mouse thymus. This protein was detected with the C-terminal α1300 but not the α23 N-terminal CDP/Cux antibody.

In electrophoretic mobility shift assays (EMSA), nuclear extracts from transfected NIH3T3 cells generated a retarded complex which could be supershifted with the α1300 CDP/Cux antibody but not with an unrelated antibody. When NIH3T3 cells were transfected with a vector expressing p75 with an influenza virus hematoglutinin (HA) tag at its carboxy-terminus, a specific signal was detected by indirect immunofluorescence in the nucleus of transfected NIH3T3 cells. These results demonstrate that I20-mRNA codes for a CDP/Cux protein of 75 kDa that localizes to the nucleus and binds to DNA.

The CDP/Cux p110 isoform contains CR2, CR3 and HD while the p75 isoform contains CR3 and HD. DNA binding properties of the two isoforms were compared using bacterially-expressed, his-tagged fusion proteins and nuclear extracts from transfected mammalian cells. The purified p110 and p75 his-tagged proteins exhibited similar DNA binding affinities, with apparent dissociation constants of 0.7×10⁻⁹ M and 1.1×10⁻⁹ M, respectively. In contrast, the DNA binding kinetics of p110 and p75 were different; p75 bound more stably to DNA than p110. In agreement with these findings, the off rate of p110 and p75 were 0.8 and 6.15 minutes, respectively. These results indicate that the p75 CDP/Cux isoform makes a more stable interaction with DNA than the p110 isoform that is expressed in S phase. Transcriptional regulation properties of p75 and p110 CDP/Cux were analyzed in parallel using reporter assays. The results from several experiments indicated that both p110 and p75 proteins repressed the p21^{WAF1/CIP1} reporter and stimulated expression from the DNA pol α reporter in a similar manner. Thus, p75 CDP/Cux localizes to the nucleus, binds to DNA, and is able to regulate transcription of target genes.

Expression of the full-length and I20-mRNAs of CDP/Cux were analyzed in a panel of breast tumor cell lines and in human mammary epithelial cells (HMEC). A fragment corresponding to the I20-mRNA was detected in MCF7, SkBr3, BT20, MDA436, MDA231, MDA468, and MCF10A but not BT549, MDA435s, or MCF12A breast tumor cell lines as determined by RNase protection analysis. In RT-PCR assays, a fragment corresponding to I20-mRNA was detected in all breast tumor cell lines analyzed (MCF12A, MCF10A, MCF7, BT20, MDA231, MDA436, SkBr3, T47D and ZR-75-1). Similarly, p75 protein was detected in MDA231, MCF10A, T47D and MCF7 breast tumor cell lines but was not in HMEC cells as determined by western blot analysis. Expression of I20-mRNA in two pairs of cell lines was further analyzed. Expression of the I20-mRNA was 3-4 fold higher in the tumorigenic Hs578T cell line than in its non tumorigenic counterpart, Hs578Bst (Hackett, et al. (1977) J. Natl. Cancer Inst. 58:1795-1806). A similar comparison in a pair of immortalized and notch-transformed mammary epithelial cell lines of mouse origin, HC11 and notch-HC11 (Dievart, et al. (1999) Oncogene 18:5973-5981), also showed that the mouse CDP/Cux I20-mRNA was expressed at a higher level (approximately two-fold) in the transformed line. Thus, these findings indicate that expression of the CDP/Cux I20-mRNA and p75 protein is activated in many breast cancer cells.

T47D cell lines stably expressing p75 were evaluated to determine whether p75 confers to mammary epithelial cells properties that are associated with cellular transformation. Although T47D cells are derived from a breast tumor, they retain the capability to differentiate and form tubules in collagen (Keely (2001) Methods Enzymol. 333:256-266; Keely, et al. (1995) J. Cell Sci. 108:595-607). Therefore, these cells provide a cellular model for investigation of the effect of putative oncogenes. Interestingly, T47D clones expressing p75 could no longer form tubules in collagen. Moreover, the colonies generated by the T47D clones expressing p75 were not hollow cysts but instead compact aggregates of cells which were devoid of a central lumen. These results indicate that upon forced expression of CDP/Cux p75, T47D cells lose their ability to form an organized epithelial sheet.

The I20-mRNA is expressed in many breast carcinomas but not in normal breast tissue. Using RT-PCR analysis, the I20-mRNA was not detected in RNA isolated from a reduction mammoplasty tissue sample from a woman without known breast pathology. This result is in accordance with the findings that the I20-mRNA was not expressed in normal mouse mammary glands. However, a strong I20-mRNA signal was observed in three cases of breast cancer, C8921D, A168A and C8961B tumors. Two of these tumors, C8921D and C8961B were invasive lobular carcinomas, whereas A168A was classified as an invasive mixed ductal lobular carcinoma but with a very diffuse growth pattern. All other tumors showing low (C9978B, C9991B, A35C, A67A, B11305D, C10544B, C10544B,

C8008A, C8644A and C8996D tumors) or no (C9096A and C7903A tumors) 120-mRNA expression were classified as ductal carcinomas.

I20-mRNA expression appears to be associated with a more diffuse growth pattern. Therefore, I20-mRNA expression was examined in an expanded panel of invasive carcinomas that were selected on the basis of their classification as either ductal, lobular or mixed lobular/ductal carcinomas. Higher I20-mRNA expression levels was significantly associated with invasive lobular and invasive mixed lobular/ductal carcinomas compared to invasive ductal carcinomas (Table 1).

TABLE 1

| | | Standard | |
|---|---|---|---|
| Carcinoma Type | Mean | Deviation | n |
| Lobular and Mixed | 115200 | 84770 | 20 |
| Ductal | 45510 | 43360 | 21 |

*p = 0.0137, Mann Whitney test (Mann and Whitney (1947) Annals Math. Stat. 18: 50-60).

These results indicate that the 120-mRNA is expressed at a higher level in a subset of breast tumors that exhibit a more diffuse growth pattern compared to tumors that exhibit the ability to form cohesive clusters and tubules. These results are in agreement with the tissue culture assays showing that mammary epithelial cells lose their ability to form tubules in collagen upon forced expression of the I20-mRNA.

I20-mRNA and p75 are expressed in acute myeloid leukemia (AML) cell lines. CDP/Cux α1300 antibody, in western blot analysis, detected the presence of p75 in four AML cell lines, KG-1a, MV4-11, RS4-11 and TF1. Likewise, a fragment corresponding to the I20-mRNA was detected in AML cell lines KG-1a, RS4-11 and TF1 but not KG-1, MV4-11, HL-60, or HEL as determined by RT-PCR analysis. These results indicate that truncated CDP/Cux isoforms are useful for detecting carcinomas as well as other classes of cancer.

Tumors and tumor cell lines have elevated levels of p110, p110, p75 and RNA transcript encoding p75 (I20-mRNA). In particular, tumors exhibiting a more diffuse growth pattern have elevated levels of a truncated CDP/Cux isoform. Accordingly, these truncated CDP/Cux proteins and nucleic acid sequences may be used as part of a diagnostic, prognostic, or predictive method or assay whereby patients may be tested for increased or elevated levels of p110, p110, p75 or RNA transcript encoding p75. Using the diagnostic method of the invention the skilled clinician will be able to determine the presence of cancer, determine the degree of invasiveness or stage of cancer, evaluate the probability of developing metastases, and predict the response to various therapeutic regimens.

In general, an assay for detecting a truncated CDP/Cux isoform comprises isolating a sample such as a biopsy sample, tissue, cell or fluid (e.g., whole blood or plasma) from a subject having or suspected of having cancer. Levels of p110, p110, p75 or p75 RNA transcript (I20-mRNA) are then evaluated in accordance with the methods provided herein. Cancers which may be detected include, but are not limited to, cancers of the brain (glioblastomas, medulloblastoma; astrocytoma, oligodendroglioma, ependymomas), lung, liver, spleen, kidney, pancreas, small intestine, blood cells, lymph nodes, colon, rectum, breast, endometrium, stomach, prostate, testicle, ovary, uterus, skin, head and neck, esophagus, bone marrow, blood or other tissue. In particular, the methods of the present invention are useful for detecting carcinomas, for example, basal and squamous cell carcinomas, gastrointestinal carcinomas (e.g., colorectal cancer, carcinoma of the esophagus, and pancreatic, hepatic, and biliary carcinomas) and breast, uterine, ovarian, testicular, prostate, and bladder carcinomas. In a preferred embodiment, the methods of the invention are used in the analysis of breast cancer.

One aspect of the present invention provides a method for detecting, in a sample, RNA transcript encoding the p75 isoform of CDP/Cux. Typically, nucleic acids are isolated from cells contained in the sample, according to standard methodologies (e.g., Sambrook et al. (1989) Molecular Cloning, a Laboratory Manual, Cold Spring Harbor Laboratories, New York). Ideally, RNA is prepared following microdissection of the tumor in order to isolate tumor cells from the normal cells present in the sample. The nucleic acid may be whole cell RNA or fractionated to Poly-A+. It may be desired to convert the RNA to a complementary DNA (cDNA). Normally, the nucleic acid is amplified.

A variety of methods may be used to evaluate or quantitate the level of p75 RNA transcript (I20-mRNA) present in the nucleic acids isolated from a sample. For example, levels of p75 RNA transcript (I20-mRNA) may be evaluated using well-known methods such as northern blot analysis (see, e.g., Sambrook et al. (1989) Molecular Cloning, a Laboratory Manual, Cold Spring Harbor Laboratories, New York); oligonucleotide or cDNA fragment hybridization wherein the oligonucleotide or cDNA is configured in an array on a chip or wafer; or RNase protection analysis or RT-PCR, as exemplified herein.

Suitable primers, probes, or oligonucleotides useful for such detection methods are exemplified herein or may be generated by the skilled artisan from the sequence provided as SEQ ID NO:1 which comprises nucleic acid sequences contained within the p75 RNA transcript (120-mRNA), but absent from the transcript of the larger CDP/Cux isoforms, i.e, p110, p110, and p200. The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred. Probes are defined differently, although they may act as primers. Probes, while perhaps capable of priming, are designed for binding to the target DNA or RNA and need not be used in an amplification process. In a preferred embodiment, the probes or primers are labeled with, for example, radioactive species (³²P, ¹⁴C, ³⁵S, ³H, or other label) or a fluorophore (rhodamine, fluorescein). Depending on the application, the probes or primers may be used cold, i.e., unlabeled, and the RNA or cDNA molecules are labeled.

Various RT-PCR methodologies may be employed to evaluate the level of p75 RNA transcript present in a sample. As clinical samples are of variable quantity and quality a relative quantitative RT-PCR reaction may be performed with an internal standard. The internal standard may be an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other assays may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

Specifically contemplated by the present invention are chip-based DNA technologies. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see, e.g., Pease, et al. (1994) Proc. Natl. Acad. Sci. USA 91(11):5022-6; Fodor, et al. (1991) Science 251(4995):767-73).

Depending on the format, detection may be performed by visual means (e.g., ethidium bromide staining of a gel). Alternatively, the detection may involve indirect identification of the product via chemiluminescence, radiolabel or fluorescent label or even via a system using electrical or thermal impulse signals (Bellus (1994) J. Macromol. Sci. Pure Appl. Chem. A311:1355-1376).

Another aspect of the invention provides a method of detecting, in a sample, truncated isoforms of CDP/Cux such as p75, p100 and p110. Accordingly, antibodies which specifically recognize p75, p100 and p110 are provided. An antibody is said to specifically recognize a truncated CDP/Cux isoform if it is able to discriminate between the various CDP/CUX isoforms (i.e., p75, p100, p110, and p200) and bind to only one isoform to form an antigen-antibody complex. For example, an antibody which specifically recognizes p75 will only bind to p75 and not p100, p110, or p200. Likewise, an antibody which specifically recognizes p110 will only bind to p110 and not to p75, p100, or p200.

Antibodies which specifically recognize a truncated isoform of CDP/Cux may be either polyclonal or monoclonal. Moreover, such antibodies may be natural or partially or wholly synthetically produced. All fragments or derivatives thereof which maintain the ability to specifically bind to and recognize a truncated isoform of CDP/Cux are also included. The antibodies may be a member of any immunoglobulin class, including any of the human classes: IgG, IgM, IgA, IgD, and IgE. Derivatives of the IgG class, however, are preferred in the present invention.

Antibody fragments may be any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. The antibody fragment may be produced by any means. For instance, the antibody fragment may be enzymatically or chemically produced by fragmentation of an intact antibody or it may be recombinantly produced from a gene encoding the partial antibody sequence. The antibody fragment may optionally be a single-chain antibody fragment. Alternatively, the fragment may comprise multiple chains which are linked together, for instance, by disulfide linkages. The fragment may also optionally be a multi-molecular complex. A functional antibody fragment will typically comprise at least about 50 amino acids and more typically will comprise at least about 200 amino acids.

The antibodies of the present invention may be generated using classical cloning and cell fusion techniques. For example, the antigen of interest is typically administered (e.g., intraperitoneal injection) to wild-type or inbred mice (e.g., BALB/c) or transgenic mice which produce desired antibodies, or rats, rabbits or other animal species which can produce native or human antibodies. The antigen may be p75, p100, p110 or fragments thereof. In a preferred embodiment, the antigen is the novel N-terminus of p75, p100 or p110. The antigen can be administered alone, or mixed with adjuvant, or expressed from a vector (VEE replicon vector), or as DNA, or as a fusion protein to induce an immune response. Fusion proteins comprise the peptide against which an immune response is desired coupled to carrier proteins, such as histidine tag (his), mouse IgG2a Fc domain, β-galactosidase, glutathione S-transferase, keyhole limpet hemocyanin (KLH), or bovine serum albumin, to name a few. In these cases, the peptides serve as haptens with the carrier proteins. After the animal is boosted, for example, two or more times, the spleen is removed and splenocytes are extracted and fused with myeloma cells using the well-known processes (Kohler and Milstein (1975) Nature 256:495-497; Harlow and Lane (1988) Antibodies: A Laboratory Manual, Cold Spring Harbor Laboratory, New York). The resulting hybrid cells are then cloned in the conventional manner, e.g., using limiting dilution, and the resulting clones, which produce the desired monoclonal antibodies, are cultured.

Alternatively, antibodies which specifically recognize a truncated isoform of CDP/Cux are derived by a phage display method. Methods of producing phage display antibodies are well-known in the art (e.g., Huse, et al. (1989) Science 246(4935):1275-81).

Selection of CDP/Cux isoform-specific antibodies is based on binding affinity to a truncated isoform of CDP/Cux and may be determined by various well-known immunoassays including, enzyme-linked immunosorbent, immunodiffusion chemiluminescent, immunofluorescent, immunohistochemical, radioimmunoassay, agglutination, complement fixation, immunoelectrophoresis, and immunoprecipitation assays and the like which may be performed in vitro, in vivo or in situ. Such standard techniques are well-known to those of skill in the art (see, e.g., “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904). Antibodies may then be purified by immunoadsorption to antigen followed by two cycles of immunodepletion with a bridging peptide corresponding to intact CDP/Cux.

Once fully characterized for specificity, the antibodies may be used in diagnostic, prognostic, or predictive methods to evaluate the levels of p75, p100 or p110 isoforms in healthy and diseased tissues via techniques such as ELISA, western blotting, or immunohistochemistry. The use of these antibodies provides a screen for the presence or absence of malignancy, as a predictor of future cancer, or for identifying the histological class or type of the cancer. Moreover, this method may be used alone or in combination with other well-known diagnostic methods for tumors. For example, immunohistochemical analysis of breast tumor sections using anti-p75, anti-p100 or anti-p110 antibodies may be added to the current procedure for diagnostic characterization of breast tumors which involves the immunohistochemical analysis of tumor sections for erbB2 expression.

The general method for detecting truncated CDP/Cux isoforms provides contacting a sample with an antibody which specifically recognizes a truncated isoform of CDP/Cux. The antibody is allowed to bind to the truncated CDP/Cux isoform to form an antibody-antigen complex. The conditions and time required to form the antibody-antigen complex may vary and are dependent on the sample being tested and the method of detection being used. Once non-specific interactions are removed by, for example, washing the sample, the antibody-antigen complex is detected using any one of the immunoassays described above as well a number of well-known immunoassays used to detect and/or quantitate antigens (see, for example, Harlow and Lane (1988) supra). Such well-known immunoassays include antibody capture assays, antigen capture assays, and two-antibody sandwich assays.

Immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques (e.g., Kennedy, et al. (1976) Clin. Chim. Acta 70:1-31; Schurs, et al. (1977) Clin. Chim Acta 81:1-40) and methods of detecting these labels are also well-known to the skilled artisan. After detecting the levels of p75, p100, p110 or p75 RNA transcript present in a sample, the results seen in a given patient are compared with a known standard. A known standard may be a statistically significant reference group of normal patients and patients that have cancer to provide diagnostic, prognostic, or predictive information pertaining the patient from whom the sample was obtained. The standard may be generated by performing prognostic analyses of multiple tumor samples derived from multiple classes of tumors. For example, a known standard for a breast tumor may comprise various clinical and biological parameters including histologic types (aveolar or ductal), tumor grades, lymph node infiltration, labeling index (or tumor cell proliferation), erbB2 expression levels, estrogen or progesterone receptor and p53 status, disease-free survival and overall survival rates.

The present invention also provides kits which are useful for carrying out the present invention. The present kit comprises a container containing an antibody which specifically recognizes a truncated CDP/cux isoform. The kit also comprises other solutions necessary or convenient for carrying out the invention. The container can be made of glass, plastic or foil and can be a vial, bottle, pouch, tube, bag, etc. The kit may also contain written information, such as procedures for carrying out the present invention or analytical information, such as the amount of reagent contained in the first container. The container may be in another container, e.g., a box or a bag, along with the written information.

The invention is described in greater detail by the following non-limiting examples.

### EXAMPLE 1

### RNA Preparation, RNase Mapping, Reverse Transcriptase-PCR

RNA was prepared using TRIZOLT (Gibco BRL, Gaithersburg, Md.) according to manufacturer's instructions and treated with RNase-free DNase at 37° C. for 30 minutes.

Riboprobes for RNase mapping were prepared using well-known methods (Zeng, et al. (2000) Gene 241:75-85). Forty μg of total RNA was annealed to 8×10⁵ cpm of labeled riboprobe at 54° C. for 16 hours in 80% formamide, 0.4 M NaCl, 0.4 M piperazine-N,N-bis (2-ethanesulfonic acid) (PIPES) (pH 6.4), 1 mM EDTA. RNA-RNA hybrids were digested with 30 units of RNase T2 (Gibco BRL, Gaithersburg, Md.) per ml at 37° C. for one hour. Hybrids were then precipitated with 20 μg of tRNA, 295 μl 4 M guanidine thiocyanate and 590 μl isopropanol. Pellets were resuspended in 80% formamide, 1×TBE and 0.1% xylene cyanol+bromophenol blue, denatured and electrophoresed on 4% acrylamide-8M urea gel.

Reverse transcriptase-polymerase chain reaction (RT-PCR) was performed on Human Multiple Tissue cDNA (MTC™) of normalized, first-strand CDNA preparations derived from different adult human tissues (Clontech, Palo Alto, Calif.). cDNA from mouse tissues, thymocytes, breast tumor cell lines and breast tumor samples (Manitoba Breast Tumor Bank, Canada) were prepared using Superscrip™ II RNaseH-Reverse transcriptase (Gibco BRL, Gaithersburg, Md.) according to the manufacturer's instructions. Primers used include:

Fi20 5′-GCTATTTTCAGGCACGGTTTCTC (SEQ ID NO:3) (human, nt −40 to −18 within intron 20, nt 1962-1984 of SEQ ID NO:1);

B22 5′-TCCACATTGTTGGGGTCGTTC (SEQ ID NO:4) (human, nt 3630-3609 of accession no: M74099; mouse, nt 3345-3324 of accession no: NM_{—}009986);

F19 5′-AGAAAGGCCGGAACCCTTCA (SEQ ID NO:5) (human, nt 3021-3041 of accession no: M74099);

Fi20m 5′-CGACGGTCCCCTTCTGGAATGG (SEQ ID NO:6) (mouse, nt −111 to −88 within intron 20);

and F18 5′-CAAGCGCTGAGTCCC (SEQ ID NO:7) (mouse, nt 2406-2420 of accession no: NM_{—}009986).

Primers were labeled in a final volume of 50 μl, containing 5 μl of 10 × kinase buffer (70 mM Tris-HCl, pH 7.6, 10 mM MgCl₂ and 5 mM DTT), 15 units of T4 polynucleotide kinase and 0.8 mCi γ³²P-ATP and incubated at 37° C. for 1 hour. Unincorporated nucleotides were removed using a Sephadex G25 spin column. PCR was performed in a final volume of 30 μl, containing 1 ng cDNA, 1.5 mM MgCl₂, 3 μl standard 10×PCR buffer (200 mM Tris-HCl, pH 8.4, 500 mM KCl), 0.45 μM of each primer, 0.12 mM dNTPs, and 1 unit of Taq polymerase (Gibco BRL, Gaithersburg, Md.). An initial denaturation step of 4 minutes at 95° C. was followed by 25 cycles of denaturation at 95° C. for 45 seconds, annealing at 61° C. for 50 seconds, and extension at 72° C. for 60 seconds, followed by a final extension at 72° C. for 7 minutes. Pilot reactions were conducted to ensure that the reaction conditions did not reach a plateau.

### EXAMPLE 2

### Cell Culture

HeLa, HEL, 293 and NIH3T3 cells were cultured in Dulbecco's Modification of Eagle's Medium (DMEM) supplemented with 10% fetal bovine serum (FBS). The breast tumor cell lines MCF7, MDA231, MDA468, T47D, Hs578T, MDA435s, and BT549 were cultured in DMEM supplemented with 5% FBS. SkBr3 cells were cultured in DMEM supplemented with 10% FBS. MDA436 cells were cultured in Leibovitz medium supplemented with 15% FBS and 10 mg/ml insulin (Gibco BRL, Gaithersburg, Md.). MCF10A and MCF12A cells were cultured in 50% DMEM-F12 medium supplemented with 5% heparin sulfate, 10 mg/ml insulin, 0.5 mg/ml hydrocortisol (SIGMA™-Aldrich, St. Louis, Mo.), 0.1 mg/ml cholera enterotoxin (Gibco BRL, Gaithersburg, Md.), and 20 ng/ml EGF (Boehringer Mannheim, Germany). HMEC cells were cultured using the manufacturer's medium and instructions (Clonetics, San Diego, Calif.). Transfections were done using ExGene500 (MBI Fermentas, Germany) according to manufacturer's instructions.

### EXAMPLE 3

### Plasmid Construction

For the expression of human intron 20-mRNA, PCR-amplification was performed using at the 5′ end a primer that contains XhoI and NotI sites linked to sequences from intron 20 (5′-ACTGCTCGAGCGGCCGCTTTTAGCAGAATGCCCTCATG, SEQ ID NO:8) and at the 3′ end a primer corresponding to nt 3862-3841 of HSCDP (accession number M74099)(5′-GTTTTTGGTGACGGGTATGGC, SEQ ID NO:9). The product was digested with XhoI and BstXI (nt 3625 of HSCDP) and ligated together with a BstXI-NotI fragment that includes nt 3625 to 4551 of HSCDP. A NotI fragment was then introduced into the corresponding site of the pcDNA3.1 vector (INVITROGEN™, Carlsbad, Calif.), and a XhoI-NotI fragment was inserted into the pMX139 vector.

### EXAMPLE 4

### T47D Collagen Assay

T47D cells were transfected with 10 μg of pMX or pMX-p75 along with 1 Ag of pSV-NEO. Cell lines stably expressing proteins were selected for 3 weeks with 400 μg/ml G418 (Gibco BRL, Gaithersburg, Md.). Assays for detecting tubule formation were performed by adding 2×10⁵ cells/ml into 1.3 mg/ml of collagen in DMEM supplemented with 5% FBS (Keely (2001) supra; Keely, et al. (1995) supra). Cells were cultured for 10 days. Tubules were visualized using a Retiga 1300 digital camera (QIMAGING™, Burnaby, BC, Canada) and a Zeiss AxioVert 135 microscope with a 10× objective (Carl Zeiss Canada Ltd., Toronto, Canada). Cells in collagen were then fixed in 4% paraformaldehyde, embedded in paraffin and sectioned (8 μM). Sections were stained with hematoxylin and eosin. Images were acquired using a PixCell II™ LCM system (Arcturus Engineering Inc., Mountain View, Calif.) using a 40× objective.

### EXAMPLE 5

### p75 Localization and Binding Assays

Nuclear extracts were prepared using well-known methods (Moon, et al. (2000) supra).

Mouse thymus protein extracts were prepared by homogenization in buffer X (50 mM Hepes, pH 7.9, 0.4 M NaCl, 4 mM NaF, 4 mM NaVO₃, 0.2 mM EDTA, 0.2 mM EGTA, 0.1% NP-40, 10% glycerol, and protease inhibitors (Roche, Indianapolis, Ind.)).

The pET-15b-based (NOVAGEN®, Madison, Wis.) bacterial expression vectors expressing CR2CR3HD and CR3HD were introduced into the BL21(DE3) E. coli and induced with IPTG. The fusion proteins were purified by affinity chromatography according to the manufacturer's instructions.

Electrophoretic mobility shift assays (EMSA) were performed, and kinetics and affinity of DNA binding measured using well-known methods (Moon, et al. (2000) supra; Moon, et al. (2001) supra).

Luciferase assays was performed using standard techniques (Moon, et al. (2001) supra).

NIH3T3 cells were plated on a coverslip and transfected with 5 μg of pMX-p75-HA. After two days, cells were fixed with 100% methanol for two minutes. After two washes with 1× phosphate-buffered saline (PBS), cells were quenched for 10 minutes in 50 mM NH₄Cl, solubilized for 10 minutes (95% PBS+5% FBS+0.5% TRITON® X-100) and incubated with anti-HA antibody (1:10000) for 1 hour at room temperature. After extensive washing, the secondary antibody (anti-mouse alexa 488-conjugated, 1:1000) was incubated for 30 minutes at room temperature in the dark. Cells were visualized using a Retiga 1300 digital camera (QIMAGING™, Burnaby, BC, Canada) and a Zeiss AxioVert 135 microscope with a 63× objective. Images were analyzed using Northern Eclipse version 6.0 (Empix Imaging, Missisauga, Canada).

### EXAMPLE 6

### Human Breast Cancer Specimen Analysis

Forty-one invasive ductal carcinomas was selected for analysis with two subgroups (Manitoba Breast Tumor Bank, Canada). All cases were processed uniformly to produce matched mirror image paraffin and frozen tissue blocks. Tumor pathology and characteristics were assessed directly in high quality paraffin sections from tissue immediately adjacent to frozen tissue sections used for RNA extraction and RT-PCR analysis (Hiller, et al. (1996) Biotechniques 21:38-44). The first group comprised invasive ductal carcinomas (n=21) showing large cohesive clusters of tumor cells forming nests or glandular arrangements, without a diffuse or infiltrating growth pattern. The second subgroup (n=21) comprised invasive tumors selected for a diffuse infiltrating growth pattern. These included ‘Mixed Ductal & Lobular Carcinomas’ (n=9) with a significant lobular component or a ‘lobular’ pattern of growth but with either focal glandular formation and/or ductal type cytological features and invasive lobular carcinomas (n=11) (Pereira, et al. (1995) Histopathology 27:219-226; Ellis, et al. (1992) Histopathology 20:479-489).

What is claimed is:

1. A method for diagnosing the presence or stage of cancer comprising detecting the level of a truncated CCAAT-displacement protein/Cut homeobox isoform in a sample wherein increased levels of a truncated CCAAT-displacement protein/Cut homeobox isoform is indicative of the presence or stage of cancer.

2. The method of claim 1 wherein the truncated CCAAT-displacement protein/Cut homeobox isoform comprises a proteolytically processed isoform of p200.

3. The method of claim 2 wherein the proteolytically processed isoform of p200 comprises p100 or p110.

4. The method of claim 1 wherein the truncated isoform of CCAAT-displacement protein/Cut homeobox comprises p75 or the RNA transcript encoding p75 polypeptide.

5. The method of claim 1 wherein detecting the level of a truncated CCAAT-displacement protein/Cut homeobox isoform comprises contacting a sample with an antibody which specifically recognizes a truncated CCAAT-displacement protein/Cut homeobox isoform so that said antibody binds to the truncated CCAAT-displacement protein/Cut homeobox isoform; detecting bound antibody; and comparing levels of the truncated CCAAT-displacement protein/Cut homeobox isoform to a known standard.

6. An antibody which specifically recognizes a proteolytically processed isoform of CCAAT-displacement protein/Cut homeobox p200.

7. The antibody of claim 6 wherein the proteolytically processed isoform of CCAAT-displacement protein/Cut homeobox p200 comprises p100 or p110.

8. An antibody which specifically recognizes the p75 isoform of CCAAT-displacement protein/Cut homeobox.

9. The method of claim 1 wherein detecting the level of a CCAAT-displacement protein/Cut homeobox isoform comprises evaluating the level of RNA transcript encoding a p75 polypeptide and comparing p75 RNA transcript levels in a sample with a known standard.

10. A kit for detecting the presence of a CCAAT-displacement protein/Cut homeobox isoform comprising an antibody which specifically recognizes a CCAAT-displacement protein/Cut homeobox isoform.
